(19) United States
(12) Patent Application Publication
(10) Pub. No.: US 2007/0286824 A1
(43) Pub. Date: Dec. 13, 2007

(54) Bleed-resistant colored microparticles and skin care compositions comprising them

(76) Inventors: Thomas Elliot Rabe, Baltimore, MD (US); Paul Graham Wildgust, Baltimore, MD (US); Christopher Todd Morrissey, Mason, OH (US); Stephen Ray Jones, Pomona, NY (US); Bryan David Grey, Bradford (GB); Paul Michael Dymond, Leeds (GB); Mark Christopher Baxter, Bradford (GB); Christina Ligia Andrianov, Monroe, NY (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/448,353

(22) Filed: Jun. 7, 2006

(51) Int. Cl. A61K 8/18; A61K 8/89

(52) U.S. Cl. 424/59; 424/401; 424/64; 424/63; 424/70.7

(57) ABSTRACT

Bleed-resistant microparticles comprising at least one colorant, a process to produce them, compositions containing them and their use in skin care applications to produce a natural, textured tone effect.

### FIELD OF THE INVENTION

This invention relates to bleed-resistant microparticles comprising at least one colorant, to a process to produce them, to compositions containing them and to their use. More particularly this invention relates to a unique process to produce bleed-resistant microparticles comprising at least one colorant, to the resulting bleed-resistant microparticles per se, to compositions containing them and to their use in skin care applications.

### BACKGROUND OF THE INVENTION

The use of finely divided colorant materials in various products within the facial cosmetics industry is well known. In areas such as facial foundations and cosmetics around the eye region, the colorants used are pigments (usually inorganic metal oxides), whose low solubility limits color release onto the skin and clothing. However, the use of these pigments limits the color palette available to cosmetic producers and does not cover the entire color palette needed to deal with the various ethnicities within a global market.

While the use of organic dyes within such areas of the color cosmetics market provides a much greater variety of color choices, such use requires the resolution of issues of controlled colorant placement and sustainability. A number of approaches to attain this have been tried.

One approach is to make the dye exhibit the solubility characteristics of a pigment. Commercially, this is done by “laking” the dye, thus forming a water-insoluble salt of the dye. However, despite being effective, the process is reversible, and a soluble dye can reform from the dye-lake.

It is generally difficult to permanently retain the colorant over long periods of time and when subjected to different environments and conditions. This is true of pigments, oil soluble dyes, and water soluble dyes.

Despite the claims of low bleed properties, the microcapsules described in patents and publications have been found to gradually release the colorant, or to “bleed”, over time when tested for prolonged periods at elevated temperatures. Color bleed occurs when a dye or pigment migrates through or off of microspheres through contact with moisture and/or other ingredients in a formulation such as alcohols or glycols, surfactants, silicones, oils, preservatives, salts and other components typically found in cosmetic formulations. Leeching or bleed of the colorant in a cosmetic composition can impair the long term visual effect of the cosmetic both in the container and on the substrate.

Thus there is a need to provide microparticles with improved color bleed resistance that can be used for a variety of applications. Specifically there is a need to provide products containing entrapped or encapsulated colorants, which products retain good shatter resistance and exhibit improved bleed resistance when subjected to different environments. This is particularly a problem when employing oil soluble and water-soluble organic dyes, where it is generally difficult to permanently retain the dye. In a cosmetic composition if the dye is not permanently retained, this can impair the long-term visual effect of the cosmetic.

The microparticles according to the present invention overcome the issue of bleeding while retaining good shatter resistance. Thus solutions containing them remain substantially uncolored even after prolonged storage at elevated temperatures.

### SUMMARY OF THE INVENTION

One aspect the present invention provides a skin care composition comprising at least one colorant, wherein said colorant is entrapped in at least one microparticulate matrix polymer, wherein the entrapment of said colorant in said microparticulate matrix polymer forms a microencapsulated colorant, wherein said microparticulate matrix polymer comprises:

a. at least one first polymer formed from a mixture of monomers comprising at least one first monomer that is an ethylenically unsaturated ionic monomer and at least one second monomer that is an ethylenically unsaturated hydrophobic monomer capable of forming a homopolymer with a glass transition temperature of from about −40 to about 50° C.; and

b. at least one second polymer formed from a mixture of monomers comprising at least one first monomer that is an ethylenically unsaturated ionic monomer and at least one second monomer that is an ethylenically unsaturated hydrophobic monomer capable of forming a homopolymer with a glass transition temperature greater than about 50° C.;

wherein said microparticulate matrix polymer further comprises secondary particles distributed throughout said microparticulate matrix polymer, wherein said secondary particles comprise a hydrophobic polymer formed from at least one ethylenically unsaturated hydrophobic monomer capable of forming a homopolymer with a glass transition temperature greater than about 50° C.

The present invention also provides a method of coloring the skin/body/eyelashes that comprises application of a liquid or solid skin care formulation having an effective coloring amount of at least one microparticulate colorant, preferably a blend of at least two microparticulate colorants as described above, to at least a part of said skin/body/eyelashes.

### DETAILED DESCRIPTION OF THE INVENTION

The present invention provides bleed-resistant microparticles containing an effective coloring amount of at least one colorant in an essentially colorless polymeric matrix formed from:
(a) from about 5 to about 95 weight percent of a polymer A formed from a mixture of monomers comprising at least one first monomer which is an ethylenically unsaturated ionic monomer and at least one second monomer that is an ethylenically unsaturated hydrophobic monomer capable of forming a homopolymer with a glass transition temperature of between about −40 and about 50° C.;
(b) from about 5 to about 95 weight percent of a polymer B formed from a mixture of monomers comprising at least one first monomer which is an ethylenically unsaturated ionic monomer and at least one second monomer that is an ethylenically unsaturated hydrophobic monomer capable of forming a homopolymer with a glass transition temperature greater than about 50° C., within which are distributed polymeric secondary particles formed from one or more ethylenically unsaturated hydrophobic monomers which are the same or different from those in polymer A.

The colorants are preferably organic. The microparticles may be uncrosslinked, but are preferably crosslinked to maintain structure while in use.

In one embodiment the colorless polymeric matrix is formed from 5 to 45 weight percent of at least one polymer A and 55 to 95 weight percent of at least one polymer B, for example 5 to 25 weight percent of at least one polymer A and 75 to 95 weight percent of at least one polymer B.

The bleed-resistant microparticles comprising at least one colorant according to the invention may be produced by a process which comprises,
A) providing an aqueous phase comprising a salt of at least one polymer A,
B) combining said aqueous phase under high shear with a second aqueous phase comprising at least one polymer B, secondary polymeric particles and, optionally, a crosslinking agent, wherein aqueous phase A) and/or B) contains at least one finely divided colorant,
C) forming a water-in-oil emulsion containing the combined aqueous phases from step B) in a water-immiscible liquid phase under high shear, which emulsion optionally comprises an oil soluble additive, an amphipathic polymeric stabilizer or a mixture thereof, and
D) subjecting the emulsion to dehydration wherein water is evaporated from the aqueous particles thereby forming solid microparticles comprising at least one colorant in a matrix polymer and having secondary polymer particles distributed throughout the matrix polymer.

The polymeric particles contain at least one polymer A and at least one polymer B, both of which are formed from a blend of monomers comprising at least one first monomer which is an ethylenically unsaturated ionic monomer and at least one second monomer which is an ethylenically unsaturated hydrophobic monomer.

The at least one ionic monomer may contain either anionic or cationic groups, or alternatively may be potentially ionic, for instance in the form of an acid anhydride. The at least one ionic monomer chosen for polymer A and polymer B may be the same or different, but should both be either anionic or cationic. Preferably the at least one ionic monomer is an ethylenically unsaturated anionic or potentially anionic monomer. Non-limiting examples of suitable anionic or potentially anionic monomers include acrylic acid, methacrylic acid, ethacrylic acid, fumaric acid, maleic acid, maleic anhydride, itaconic acid, itaconic acid anhydride, crotonic acid, vinyl acetic acid, (meth) allyl sulfonic acid, vinyl sulfonic acid and 2-acrylamido-2-methyl propane sulfonic acid. Preferred anionic monomers are carboxylic acids or acid anhydrides.

When the at least one ionic monomer is anionic, for instance a carboxylic acid or anhydride, it will preferably be partially or completely neutralized with at least one volatile counterion. The volatile counterion may be ammonia or a volatile amine component. Generally the volatile amine component will be a liquid that can be evaporated at low to moderate temperatures, for instance at temperatures up to 200° C. Preferably, it will be possible to evaporate the volatile amine under reduced pressure at temperatures below 100° C. Thus the polymer may be produced in free acid form and then neutralized with an aqueous solution of ammonium hydroxide or a volatile amine, for instance ethanolamine, methanolamine, 1-propanolamine, 2-propanolamine, dimethanolamine or diethanolamine. Alternatively the polymer may be prepared by copolymerizing the ammonium or volatile amine salt of an anionic monomer with the hydrophobic monomer.

During the dehydration step D) at least a part of the at least one volatile counterion component of the salt is desirably evaporated. For instance, where the polymeric counterion is the ammonium salt, at least a part of the volatile component ammonia will be evaporated. Consequently, during the distillation stage the polymer will be converted to its free acid or free base form.

Both polymer A and polymer B are copolymers formed from a blend of monomers comprising at least one first monomer which is an ethylenically unsaturated ionic monomer and at least one second monomer which is an ethylenically unsaturated hydrophobic monomer. The polymers differ with respect to the hydrophobic monomer. Thus, in polymer A the ethylenically unsaturated hydrophobic monomer is selected from those capable of forming a homopolymer with a glass transition temperature between −40 and 50° C., while in polymer B the ethylenically unsaturated hydrophobic monomer is selected from those capable of forming a homopolymer with a glass transition temperature greater than 50° C. Its glass transition temperature is preferably at least 60° C. or even at least 80° C.

Specific non-limiting examples of hydrophobic monomers capable of forming a homopolymer with a glass transition temperature between −40 and 50° C. include C₁-C₈alkyl acrylates such as methyl acrylate, ethyl acrylate, propyl acrylate, isopropyl acrylate and the various isomers of butyl acrylate, amyl acrylate, hexyl acrylate and octyl acrylate, such as 2-ethylhexyl acrylate. Other examples include C₄-C₈alkyl methacrylates such as n-butyl methacrylate, n-hexyl methacrylate, n-octyl methacrylate, 2-ethylhexyl methacrylate, and alkenes such as propylene and n-butylene.

Specific non-limiting examples of hydrophobic monomers capable of forming a homopolymer with a glass transition temperature greater than 50° C. include styrene, methyl methacrylate, ethyl methacrylate, isopropyl methacrylate, tertiary butyl methacrylate, cyclohexyl methacrylate, phenyl methacrylate and isobomyl methacrylate. Other possibilities include using modified styrenics or other methacrylate and acrylate esters, provided the monomers produce polymers having a glass transition temperature (Tg) greater than 50° C.

Generally, polymers A and B may be prepared by any suitable polymerization process. For instance the polymers can be conveniently prepared by aqueous emulsion polymerization for instance as described in EP-A-697423 or U.S. Pat. No. 5,070,136. The polymers can then be neutralized by the addition of an aqueous solution of ammonium hydroxide or a volatile amine.

In a typical polymerization process, a blend of at least one hydrophobic monomer and at least one ionic monomer is emulsified into an aqueous phase which contains a suitable amount of at least one emulsifying agent. Typically, the at least one emulsifying agent may be any commercially available emulsifying agent suitable for forming an aqueous emulsion. Desirably these emulsifying agents will tend to be more soluble in the aqueous phase than in the water immiscible monomer phase and thus will tend to exhibit a high hydrophilic lipophilic balance (HLB). Emulsification of the monomer mixture may be effected by known emulsification techniques, including subjecting the monomer/aqueous phase to vigorous stirring or shearing or alternatively passing the monomer/aqueous phase through a screen or mesh. Polymerization may then be effected by use of a suitable initiator system, for instance at least one UV initiator or thermal initiator. A suitable technique of initiating the polymerization would be to elevate the temperature of an aqueous emulsion of the monomers to above 70 or 80° C. and then add between 50 and 1000 ppm of ammonium persulfate by weight of monomer.

Generally the polymer A has a molecular weight of up to 100,000 (determined by GPC using standard industrial parameters). Preferably the polymer has a molecular weight of below 50,000, for instance 10,000 to 30,000. In particular the molecular weight for polymer A is around 5,000 to 15,000.

A particularly preferred polymer A is a terpolymer of ethyl acrylate/methyl methacrylate with acrylic acid ammonium salt. Preferably this polymer is also used when the process employs a cross-linking agent, which is especially zinc oxide or ammonium zirconium carbonate.

Typically the monomer blend for making the polymer A may contain at least 70% by weight of at least one hydrophobic monomer, the remainder being made up of at least one ionic monomer. Generally though the hydrophobic monomer will be present in amounts of at least 80% by weight. Preferred compositions contain between 70 and 95% by weight of at least one hydrophobic polymer, for instance around 80 or 90%.

Generally the matrix polymer B has a molecular weight of up to 300,000 (determined by GPC using standard industrial parameters). Preferably the polymer has a molecular weight of below 50,000, for instance 2,000 to 20,000. In particular the molecular weight for the matrix polymer is around 4,000 to 12,000.

A particularly preferred matrix polymer B is a copolymer of styrene with ammonium acrylate. More preferably this polymer is used when the process employs a cross-linking agent, which is especially zinc oxide or ammonium zirconium carbonate.

Typically the monomer blend in for making the matrix polymer B may contain at least 50% by weight of at least one hydrophobic monomer, the remainder being made up of at least one ionic monomer. Generally though the hydrophobic monomer will be present in amounts of at least 60% by weight. Preferred compositions contain between 65 and 90% by weight of at least one hydrophobic polymer, for instance around 70 or 75%.

In an alternative version of the process, the at least one ionic monomer may be cationic or potentially cationic, for instance an ethylenically unsaturated amine. In this form of the invention the volatile counterionic component is at least one volatile acid component. Thus, in this form of the invention the polymers A and B can be formed in an analogous way to the aforementioned anionic polymers, except that the anionic monomer is replaced by a cationic or potentially cationic monomer. Generally where the polymer is prepared in the form of a copolymer of at least one free amine and at least one hydrophobic monomer, it is neutralized by adding at least one suitable volatile acid, for instance acetic acid, formic acid, propanoic acid, butanoic acid or even carbonic acid. Preferably the polymer is neutralized by acetic acid, formic acid, acid or carbonic acid.

Suitable non-limiting examples of cationic or potentially cationic monomers include dialkyl aminoalkyl(meth)acrylates, dialkyl aminoalkyl(meth)acrylamides or allyl amines and other ethylenically unsaturated amines and their acid addition salts. Typically the dialkyl aminoalkyl(meth)acrylates include dimethyl aminomethyl acrylate, dimethyl aminomethyl methacrylate, dimethyl aminoethyl acrylate, dimethyl aminoethyl methacrylate, diethyl aminoethyl acrylate, diethyl aminoethyl methacrylate, dimethyl aminopropyl acrylate, dimethyl aminopropyl methacrylate, diethyl aminopropyl acrylate, diethyl aminopropyl methacrylate, dimethyl aminobutyl acrylate, dimethyl aminobutyl methacrylate, diethyl aminobutyl acrylate and diethyl aminobutyl methacrylate. Typically the dialkyl aminoalkyl(meth)acrylamides include dimethyl aminomethyl acrylamide, dimethyl aminomethyl methacrylamide, dimethyl aminoethyl acrylamide, dimethyl aminoethyl methacrylamide, diethyl aminoethyl acrylamide, diethyl aminoethyl methacrylamide, dimethyl aminopropyl acrylamide, dimethyl aminopropyl methacrylamide, diethyl aminopropyl acrylamide, diethyl aminopropyl methacrylamide, dimethyl aminobutyl acrylamide, dimethyl aminobutyl methacrylate, diethyl aminobutyl acrylate and diethyl aminobutyl methacrylamide. Typically the allyl amines include diallyl amine and triallyl amine.

The secondary particles comprise a hydrophobic polymer that has been formed from at least one ethylenically unsaturated hydrophobic monomer which is capable of forming a homopolymer of glass transition temperature in excess of 50° C. and optionally other monomers, which hydrophobic polymer is different from polymer A and the matrix polymer B. The at least one ethylenically unsaturated hydrophobic monomer may be any of the monomers defined above in respect of the second monomer used to form the matrix polymer B. In one embodiment, the hydrophobic monomer is the same as the second monomer used to form the matrix polymer. Specific non-limiting examples of said hydrophobic monomers include styrene, methyl methacrylate, tertiary butyl methacrylate, phenyl methacrylate, cyclohexyl methacrylate and isobornyl methacrylate. In one embodiment the hydrophobic monomer is styrene.

The hydrophobic monomer may be polymerized alone or alternatively may optionally be polymerized with at least one other hydrophobic monomer as defined above. It may be possible to include other monomers that are not hydrophobic monomers capable of forming a homopolymer of glass transition temperature in excess of 50° C., provided that such monomers do not bring about any deleterious effects. The other monomer may be a hydrophobic monomer, for instance longer chain alkyl and esters of acrylic or methacrylic acid, such as 2-ethylhexyl acrylate or stearyl acrylate. Typically, where such monomers are included, they should be present in an amount of no more than 20% by weight based on the weight of monomers used for the secondary particles. Preferably, these monomers will be present in amount less than 10% by weight, for example less than 5% by weight.

Alternatively the at least one other monomer may be a hydrophilic monomer. The hydrophilic monomer may be nonionic, for instance acrylamide, or it can be ionic, for instance as defined in respect of the first monomer used to form the polymers A and B. Generally, such monomers tend to be used in smaller proportions so that the polymer remains hydrophobic. Where such monomers are included, they should be present in an amount of no more than 20% by weight based on the weight of monomers used for the secondary particles. Preferably, these monomers will be present in an amount less than 10% by weight, for example less than 5% by weight.

It is particularly preferred that the secondary particles comprise a hydrophobic polymer that has been formed entirely from ethylenically unsaturated hydrophobic monomer(s) which is/are capable of forming a homopolymer of glass transition temperature in excess of 50° C. A particularly suitable hydrophobic polymer is a copolymer of styrene and methyl methacrylate or a homopolymer of styrene. The copolymer of styrene with methyl methacrylate will generally be formed from at least 40% by weight styrene and up to 60% by weight methyl methacrylate. Preferably, the copolymer will have a weight ratio of styrene to methyl methacrylate moieties of between 50:50 to 95:5 and more preferably 60:40 to 80:20, for example 70:30 to 75:25.

Generally, the secondary particles will have an average particle size of below 1 micron, and usually below 750 nm. Preferably, the secondary particles will have an average particle size in the range between 50 and 500 nm. These secondary particles may be prepared by any conventional means. Typically, the particles may be prepared by aqueous emulsion polymerization. Preferably, the particles are prepared by aqueous microemulsion polymerization according to any typical microemulsion polymerization process documented in the prior art, for instance as described in EP-A-531005 or EP-A-449450.

Typically, the secondary particles may be prepared by forming a microemulsion comprising a continuous aqueous phase (between 20 and 80% by weight), a dispersed oil phase comprising at least one monomer (between 10 and 30% by weight), and at least one surfactant and/or stabilizer (between 10 and 70% by weight). Generally the surfactant and/or stabilizer will exist predominantly in the aqueous phase. A preferred surfactant and/or stabilizer is an aqueous solution of the polymer used to form the polymeric matrix. A particularly preferred surfactant/stabilizer is a copolymer of ammonium acrylate with styrene, as defined above in relation to the matrix polymer B.

Polymerization of the at least one monomer in the microemulsion can be effected by a suitable initiation system, for instance a UV initiator or thermal initiator. A suitable technique of initiating the polymerization is, for instance, to elevate the temperature of the aqueous emulsion of monomer to above 70 or 80° C. and then to add between 50 and 1000 ppm of ammonium persulfate or an azo compound such as azodiisobutyronitrile by weight of monomer. Alternatively, a suitable peroxide, e.g. a room-temperature curing peroxide, or a photo-initiator may be used. It may be preferred that polymerization is carried out at about room temperature, e.g. with a photoinitiator.

Generally the secondary particles comprise a polymer that has a molecular weight of up to 2,000,000 (determined by GPC using the standard industrial parameters). Preferably the polymer has a molecular weight of below 500,000, for instance 5,000 to 300,000. Usually the molecular weight for the polymeric secondary particles is between 100,000 and 200,000.

It is preferred that the secondary particles have a core shell configuration in which the core comprises the hydrophobic polymer surrounded by a polymeric shell. More preferably the secondary particles comprise a core comprising the hydrophobic polymer and a shell comprising the polymers A and B. It is particularly preferable that the shell of the polymer is formed around the core of hydrophobic polymer and during polymerization.

The polymeric products can be further enhanced if the matrix polymer is cross-linked. This cross-linking can be as a result of including a cross-linking step in the process. This can be achieved by including self cross-linking groups in the polymer, for instance monomer repeating units carrying a methylol functionality. Preferably though the cross-linking is achieved by including a cross-linking agent with the aqueous phase polymer. The cross-linking agents are generally compounds which react with functional groups on the polymer chain. For instance, when the polymer chain contains anionic groups, suitable cross-linking organic agents include aziridines, diepoxides, carbodiamides and silanes. A preferred class of organic cross-linking agents includes compounds that form covalent bonds between polymer chains, for instance silanes or diepoxides. Suitable crosslinkers also include zinc oxide, zinc ammonium carbonate, zinc acetate, and zirconium salts such as zirconium ammonium carbonate for example. A particularly preferred cross-linking agent is zinc oxide, which is both a colorant pigment and a crosslinker.

The crosslinking agent generally constitutes from 1 to 50% by weight of the encapsulated particles, preferably between 2 and 40%, and most preferably between 5 and 30%. The cross-linking process desirably occurs primarily during the dehydration step. Thus where a cross-linking agent is included, crosslinking will generally proceed only slowly until the dehydration step D) and removal of the volatile counterion is started.

In one embodiment the microparticles are coated with an oil-soluble or dispersible additive in-situ during formation of the particles through dehydration and crosslinking of a water-in-oil emulsion. Said additive is desirably present during the emulsification step. The additive adheres to the surface of the particles.

The choice of oil-soluble or dispersible additive and the amount present according to the invention will depend on the intended use of the composition and the effectiveness of the compound. In skin care applications, the oil-soluble or dispersible additive chosen is acceptable for skin contact, as is well known to the skilled formulator. Suitable oil-soluble or dispersible additives are incorporated at levels generally between 1 and 20% by weight based on the weight of the matrix bead (equivalent to 90 to 300% on weight of the colorant). Preferably 5 to 15% by weight of the oil-soluble or dispersible additive is employed.

The oil-soluble or dispersible additive may include fatty alcohols such as GUERBET alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol.

The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids).

Further components that can be used are dicarboxylic acids of C₂-C₁₂, such as adipic acid, succinic acid, and maleic acid. Aromatic carboxylic acids, saturated and/or unsaturated, especially benzoic acid, can be used.

Additional components that can be used as the oil soluble or dispersible additive include carboxylic acid salts: for example the salts of C₈-C₂₄, preferably C₁₄-C₂₀ saturated or unsaturated fatty acids, C₈-C₂₂ primary or secondary alkyl sulfonates, alkyl glycerol sulfonates, the sulfonated polycarboxylic acids described in published British Patent 1,082,179, paraffin sulfonates, N-acyl, N′-alkyl taurates, alkyl phosphates, isethionates, alkyl succinamates, alkyl sulphosuccinates, monoesters or diesters of sulfosuccinates, N-acyl sarcosinates, alkyl glycoside sulfates, polyethoxycarboxylates, the cation being an alkali metal (sodium, potassium, lithium), an unsubstituted or substituted ammonium residue (methyl, dimethyl, trimethyl, tetramethyl ammonium, dimethyl piperidinium, etc.) or a derivative of an alkanol amine (monoethanol amine, diethanol amine, triethanol amine, etc.); alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Waxes may be used herein. This includes, but is not limited to, esters of long-chain acids and alcohols as well as compounds having wax-like properties, e.g., camauba wax (Copemicia Cerifera), beeswax (white or yellow), lanolin wax, candellila wax (Euphorbia Cerifera), ozokerite, japan wax, paraffin wax, microcrystalline wax, ceresin, cetearyl esters wax, synthetic beeswax, etc.; also, hydrophilic waxes as cetearyl alcohol or partial glycerides.

Silicones or siloxanes (organosubstituted polysiloxanes) may be used herein. This includes, but is not limited to, dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form; linear polysiloxanes: dimethicones such as Dow Corning® 200 fluid, Mirasil® DM (Rhodia), dimethiconol; cyclic silicone fluids: cyclopentasiloxanes, volatiles such as Dow Coming® 345 fluid, Silbionee grade, Abil® grade; phenyltrimethicones; Dow corning® 556 fluid. Also suitable are simethicones, which are mixtures of dimethicones having an average chain length of from 200 to 300 dimethylsiloxane units with hydrogenated silicates. A detailed survey by Todd et al. of suitable volatile silicones may be found in addition in Cosm. Toil. 91, 27 (1976). Especially suitable are ethoxylated propoxylated dimethicone (e.g. Dow Corning 5225C Formulation Aid) and aminopropyldimethicone (e.g. Tinocare SiAl from Ciba Specialty Chemicals).

Fluorinated or perfluorinated alcohols and acids may be used herein. This includes, but is not limited to, perfluordodecanoic acid, perfluordecanoic acid, perfluoro-tert-butyl alcohol, perfluoroadipic acid, 2-(perfluoroalkyl)ethanol (ZONYL® BA-L).

The oil-soluble or dispersible additive may be an anionic surfactant. Examples of such anionic surfactants include:

alkyl ester sulfonates of the formula R₁₀₀—CH(SO₃M)—COOR₂₀₀, where R_{100 is} a C₈-C₂₀, preferably C₁₀-C₁₆alkyl radical, R₂₀₀ is a C₁-C₁₆, preferably C₁-C₃ alkyl radical, and M is an alkaline cation (sodium, potassium, lithium), substituted or non-substituted ammonium (methyl, dimethyl, trimethyl, tetramethyl ammonium, dimethyl piperidinium, etc.) or a derivative of an alkanol amine (monoethanol amine, diethanol amine, triethanol amine, etc.);

alkyl sulfates of the formula R₃₀₀OSO₃M, where R₃₀₀ is a C₅-C₂₄, preferably C₁₀-C₁₈ alkyl or hydroxyalkyl radical, and M is a hydrogen atom or a cation as defined above, and their ethyleneoxy (EO) and/or propyleneoxy (PO) derivatives, having on average 0.5 to 30, preferably 0.5 to 10 EO and/or PO units;

alkyl amide sulfates of the formula R₄₀₀CONHR₅₀₀OSO₃M, where R₄₀₀ is a C₂-C₂₂, preferably C₆-C₂₀ alkyl radical, R₅₀₀ is a C₂-C₃ alkyl radical, and M is a hydrogen atom or a cation as defined above, and their ethyleneoxy (EO) and/or propyleneoxy (PO) derivatives, having on average 0.5 to 60 EO and/or PO units.

The oil-soluble or dispersible additive may be a non-ionic surfactant. Nonionic surfactants that may be used include the primary and secondary alcohol ethoxylates, especially the C₈-C₂₀ aliphatic alcohols ethoxylated with an average of from 1 to 20 moles of ethylene oxide per mole of alcohol, and more especially the C₁₀-C₁₅ primary and secondary aliphatic alcohols ethoxylated with an average of from 1 to 10 moles of ethylene oxide per mole of alcohol. Non-ethoxylated nonionic surfactants include alkylpolyglycosides, glycerol monoethers, and polyhydroxyamides (glucamides).

Some particular examples of such nonionic surfactants include: polyalkoxylenated alkyl phenols (i.e. polyethyleneoxy, polypropyleneoxy, polybutyleneoxy), the alkyl substituent of which has from 6 to 12 C atoms and contains from 5 to 25 alkoxylenated units; examples are TRITON X-45, X-114, X-100 and X-102 marketed by Rohm & Haas Co., and IGEPAL NP2 to NP17 made by Rhodia; C₈-C₂₂ polyalkoxylenated aliphatic alcohols containing 1 to 25 alkoxylenated (ethyleneoxy, propyleneoxy) units; examples include TERGITOL 15-S-9, TERGITOL 24-L-6 NMW marketed by Dow, NEODOL 45-9, NEODOL 23-65, NEODOL 45-7, and NEODOL 45-4 marketed by Shell Chemical Co., KYRO EOB marketed by The Procter & Gamble Co., SYNPERONIC A3 to A9 made by ICI, RHODASURF IT, DB and B made by Rhodia; the products resulting from the condensation of ethylene oxide or propylene oxide with propylene glycol and/or ethylene glycol, with a molecular weight in the order of 2,000 to 10,000, such as the PLURONIC products marketed by BASF; the products resulting from the condensation of ethylene oxide and/or propylene oxide with ethylene diamine, such as the TETRONIC products marketed by BASF; C₈-C₁₈ ethoxyl and/or propoxyl fatty acids containing 5 to 25 ethyleneoxy and/or propyleneoxy units; C₈-C₂₀ fatty acid amides containing 5 to 30 ethyleneoxy units; ethoxylated amines containing 5 to 30 ethyleneoxy units; alkoxylated amidoamines containing 1 to 50, preferably 1 to 25 and in particular 2 to 20 alkyleneoxy (preferably ethyleneoxy) units; amine oxides such as the oxides of alkyl C₁₀-C₁₈ dimethylamines, the oxides of alkoxy C₈-C₂₂ ethyl dihydroxy ethylamines; alkoxylated terpene hydrocarbons such as ethoxylated and/or propoxylated α- or β pinenes, containing 1 to 30 ethyleneoxy and/or propyleneoxy units; alkylpolyglycosides obtainable by condensation (for example by acid catalysis) of glucose with primary fatty alcohols (e.g. those in U.S. Pat. Nos. 3,598,865 and 4,565,647; and EP-A-132 043 and EP-A-132 046) having a C₄-C₂₀, preferably C₈-C₁₈ alkyl group and an average number of glucose units in the order of 0.5 to 3, preferably in the order of 1.1 to 1.8 per mole of alkylpolyglycoside (APG), particularly those having a C₈-C₁₄ alkyl group and on average 1.4 glucose units per mole, a C₁₂-C₁₄ alkyl group and on average 1.4 glucose units per mole, a C₈-C₁₄ alkyl group and on average 1.5 glucose units per mole or a C₈-C₁₀ alkyl group and on average 1.6 glucose units per mole, marketed under the names GLUCOPON 600 EC, GLUCOPON 600 CSUP, GLUCOPON 650 EC and GLUCOPON 225 CSUP respectively and made by Henkel.

Another class of suitable surfactants comprises certain mono-long chain-alkyl cationic surfactants. Cationic surfactants of this type include quaternary ammonium salts of the general formula R₁₀R₂₀R₃₀R₄₀N⁺X⁻ wherein the R groups are long or short hydrocarbon chains; typically alkyl, hydroxyalkyl or ethoxylated alkyl groups, and X is a counter-ion (for example, compounds in which R₁₀ is a C₈-C₂₂ alkyl group, preferably a C₈-C₁₀ or C₁₂-C₁₄ alkyl group, R₂₀ is a methyl group, and R₃₀ and R₄₀, which may be the same or different, are methyl or hydroxyethyl groups); and cationic esters (for example, choline esters).

Also useful are ethoxylated carboxylic acids or polyethylene glycol esters (PEG-n acylates), linear fatty alcohols having from 8 to 22 carbon atoms, products from 2 to 30 mol of ethylene oxide and/or from 0 to 5 mol propylene oxide with fatty acids having from 12 to 22 carbon atoms and with alkylphenols having from 8 to 15 carbon atoms in the alkyl group, fatty alcohol polyglycol ethers such as Laureth-n, Ceteareth-n, Steareth-n and Oleth-n, fatty acid polyglycol ethers such as PEG-n Stearate, PEG-n Oleate and PEG-n Cocoate; polyethoxylated or acrylated lanolin; monoglycerides and polyol esters; C₁₂-C₂₂ fatty acid mono- and di-esters of addition products of from 1 to 30 mol of ethylene oxide with polyols; fatty acid and polyglycerol esters such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of these substance classes are also suitable. Fatty acid polyglycol esters such as monostearate diethylene glycol, fatty acid and polyethylene glycol esters; fatty acid and saccharose esters such as sucro esters, glycerol and saccharose esters such as sucro glycerides; sorbitol and sorbitan: sorbitan mono- and di-esters of saturated and unsaturated fatty acids having from 6 to 22 carbon atoms and ethylene oxide addition products; polysorbate-n series, sorbitan esters such as sesquiisostearate, sorbitan, PEG-(6)-isostearate sorbitan, PEG-(10)-laurate sorbitan, PEG-17-dioleate sorbitan; glucose derivatives: C₈-C₂₂ alkyl-mono and oligo-glycosides and ethoxylated analogues with glucose being preferred as the sugar component; O/W emulsifiers such as Methyl Gluceth-20 sesquistearate, sorbitan stearate/sucrose cocoate, methyl glucose sesquistearate, cetearyl alcohol/cetearyl glucoside; also W/O emulsifiers such as methyl glucose dioleate/methyl glucose isostearate.

Oil-soluble or dispersible additives also include sulfates and sulfonated derivatives: e.g. dialkylsulfosuccinates (e.g. DOSS, dioctyl sulfosuccinate), alkyl lauryl sulfonate, linear sulfonated paraffins, sulfonated tetrapropylene sulfonate, sodium lauryl sulfates, ammonium and ethanolamine lauryl sulfates, lauryl ether sulfates, sodium laureth sulfates, acetyl isothionates, alkanolamide sulfates such as taurines, methyl taurines, and imidazole sulfates; and

Oil-soluble or dispersible additives also include amine derivatives: amine salts, ethoxylated amines such as amine oxides, amines with chains containing a heterocycle such as alkyl imidazolines, pyridine derivatives, isoquinolines, cetyl pyridinium chloride, cetyl pyridinium bromide, quaternary ammonium compounds such as cetyltrimethylammonium bromide, and stearylalkonium salts; amide derivatives: alkanolamides such as acylamide DEA, ethoxylated amides, such as PEG-n acylamide, oxydeamide;polysiloxane/polyalkyl/polyether copolymers and derivatives: dimethicone, copolyols, silicone polyethylene oxide copolymers and silicone glycol copolymers; propoxylated or POE-n ethers (Meroxapols), Polaxamers or poly(oxyethylene)ₘ-block-poly(oxypropylene)ₙ-block(oxyethylene) copolymers; zwitterionic surfactants that carry at least one quaternary ammonium group and at least one carboxylate and/or sulfonate group in the molecule, zwitterionic surfactants that are especially suitable include the so-called betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines each having from 8 to 18 carbon atoms in the alkyl or acyl group and also cocoacylaminoethylhydroxyethyl-carboxy-methylglycinate, N-alkylbetaines and N-alkylaminobetaines; alkylimidazolines, alkylopeptides and lipoaminoacids; self-emulsifying bases (see K. F. DePolo—A Short Textbook Of Cosmetology, Chapter 8, Table 8-7, p 250-251); non-ionic bases such as PEG-6 Beeswax (and) PEG-6 stearate (and) polyglyceryl-2-isostearate [Apifac], Glyceryl stearate (and) PEG-100 stearate, [Arlacel 165], PEG-5 Glyceryl stearate [Arlatone 983 S], sorbitan oleate (and) polyglyceryl-3-ricinoleate [Arlacel 1689], sorbitan stearate and sucrose cocoate [Arlatone 2121], glyceryl stearate and laureth-23 [Cerasynth 945], cetearyl alcohol and Ceteth-20 [Cetomacrogol Wax], cetearyl alcohol and Polysorbate 60 and PEG-150 and stearate-20 [Polawax GP 200, Polawax NF], cetearyl alcohol and cetearyl polyglucoside [Emulgade PL 1618], cetearyl alcohol and Ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and Steareth-7 and Steareth-10 [Emulgator E 2155], cetearyl Alcohol and Steareth-7 and Steareth-10 [Emulsifying wax U.S.N.F], glyceryl stearate and PEG-75 stearate [Gelot 64], propylene glycol ceteth-3 acetate [Hetester PCS], propylene glycol isoceth-3 acetate [Hetester PHA], cetearyl alcohol and Ceteth-12 and Oleth-12 [Lanbritol Wax N 21], PEG-6 stearate and PEG-32 stearate [Tefose 1500], PEG-6 stearate and Ceteth-20 and Steareth-20 [Tefose 2000], PEG-6 Stearate and ceteth-20 and Glyceryl Stearate and steareth-20 [Tefose 2561], glyceryl stearate and Ceteareth-20 [Teginacid H, C, X]; anionic alkaline bases such as PEG-2 stearate SE, glyceryl stearate SE [Monelgine, Cutina KD] and propylene glycol stearate [Tegin P]; anionic acid bases such as cetearyl alcohol and sodium cetearyl sulfate [Lanette N, Cutina LE, Crodacol GP], cetearyl alcohol and sodium lauryl sulfate [Lanette W], Trilaneth-4 phosphate and glycol stearate and PEG-2 stearate [Sedefos 75], glyceryl stearate and sodium lauryl sulfate [Teginacid Special]; and cationic acid bases such as cetearyl alcohol and cetrimonium bromide.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents and the like.

Inorganic salts and complexes or pigments useful herein include those that may be dispersed through the oil phase to coat the encapsulated particles include for example zinc and derivatives thereof (e.g. zinc oxide, zinc ammonium carbonate, zinc acetate, zinc sulfate), zirconium and derivatives thereof (e.g. zirconium ammonium carbonate).

The microparticles may also have a polymeric amphipathic stabilizer D located at their surface. Such stabilizers are amphipathic in that they contain both hydrophobic and hydrophilic groups. By virtue of this structure, some amphipathic materials are able to be used to stabilize dispersions. The hydrophilic group is ionic or polar in nature.

In general amphipathic polymers suitable for stabilization are hydrophobic polymers prepared from monomers having Tg values between about −110° C. and 20° C. or mixtures thereof, for example C₁-C₃₀alkyl acrylates such as methyl acrylate (Tg 9° C.), ethyl acrylate (Tg −23° C.), propyl acrylate, butyl. acrylate (Tg −49° C.), etc., as well as others including but not limited to stearyl methacrylate (Tg −100° C.).

Tg values are found for example in Polymer Handbook (3rd Edition), Ed. Brandrup & immergut, Pub: Wiley Interscience, 1989 ISBN: 0-471-81244-7.

Other materials that would work include oil-soluble polymers composed of potentially anionic monomers, i.e. monomers that would become anionic in a high pH environment, and also potentially cationic monomers, i.e. monomers that would become cationic in a low pH environment, such as an amine-type molecule. Anionic monomers include acrylic acid, methacrylic acid, maleic anhydride, ethacrylic acid, fumaric acid, maleic acid? maleic anhydride, itaconic acid, itaconic acid anhydride, crotonic acid, vinyl acetic acid, (meth) allyl sulfonic acid, vinyl sulfonic acid and 2-acrylamido-2-methyl propane sulfonic acid. Preferred anionic monomers are carboxylic acids or acid anhydrides. Particularly preferred amphipathic stabilizers include those outlined in WO-2005-123009, page 15, lines 17 to 22 and WO-2005-123796, page 20, lines 19 to 26.

A preferred type of polymeric amphipathic stabilizer D is a copolymer of an alkyl(meth)acrylate and a carboxylic functional monomer which may be prepared as follows:

The alkyl(meth)acrylate, carboxylic functional monomer and a suitable oil soluble thermal initiator, for example 2,2′-azobis(2-methylbutyronitrile), are dissolved in an inert solvent, for example an aliphatic or aromatic hydrocarbon solvent such as ISOPAR G®. This mixture is fed into a vessel containing further solvent and thermal initiator over a period of 2 to 6 hours at reaction temperatures of 80 to 90° C. The reaction is maintained at this temperature for a further two hours before being cooled and discharged.

The alkyl group of the alkyl(meth)acrylate may be any suitable alkyl group, however C₁-C₂₂ alkyl groups are preferred. The carboxylic functional monomer is selected from those described previously.

The alkyl(meth)acrylate:carboxylic functional monomer ratio may be between 0.5 to 8.0:1 on a molar basis, preferably between 0.75 to 6.0:1, and most preferably between 1.0 to 4.0:1 on a molar basis.

In one embodiment of the present invention the preferred stabilizer is EMI-759 available from Ciba Specialty Chemicals.

The molecular weight may be determined by conventional chromatographic techniques well known to those skilled in the art. Typical molecular weights may be in the range of 10,000 to 60,000, most typically in the range of 15,000 to 40,000.

Generally average particle size diameters of bleed-resistant colorant microparticles up to about 400 microns are achievable according to the invention. Preferably the average particle size diameter of the bleed-resistant colorant microparticles is less than about 100 microns for skin care applications. Advantageously the average particle size diameter is in the range of about 1 to 60 microns, e.g. 1 to 40 microns and especially between 1 and 30 microns. Average particle size is determined by a Coulter particle size analyzer according to standard procedures well documented in the literature.

The particles entrap one or more colorants, and the colorant may be any colorant, for instance a dye, pigment or lake. Typical suitable colorants for cosmetics include any organic or inorganic pigment or colorant approved for use in cosmetics by CTFA and the FDA such as lakes, iron oxides, titanium dioxide, iron sulfides or other conventional pigments used in cosmetic formulations. Organic colorants are preferred.

Examples of pigments include inorganic pigments such as carbon black, D&C Red 7, calcium lake, D&C Red 30, talc lake, D&C Red 6, barium lake, russet iron oxide, yellow iron oxide, brown iron oxide, talc, kaolin, mica, mica titanium, red iron oxide, magnesium silicate and titanium oxide; and organic pigments such as Red No. 202, Red No. 204, Red No. 205, Red No. 206, Red No. 219, Red No. 228, Red No. 404, Yellow No. 205, Yellow No. 401, Orange No. 401 and Blue No. 404. Examples of vat dyes are Red No. 226, Blue No. 204 and Blue No. 201. Examples of lake dyes include various acid dyes which are laked with aluminum, calcium or barium.

In one embodiment the colorant is an aqueous solution of a water-soluble dye. Such dyes may include FD&C Blue No. 11, FD&C Blue No. 12, FD&C Green No. 13, FD&C Red No. 13, FD&C Red No. 140, FD&C Yellow No. 15, FD&C Yellow No. 16, D&C Blue No. 14, D&C Blue No. 19; D&C Green No. 15, D&C Green No. 16, D&C Green No. 18, D&C Orange No. 14, D&C Orange No.15, D&C Orange No. 110, D&C Orange No. 111, D&C Orange No. 117, FD&C Red No. 14, D&C Red No. 16, D&C Red No. 17, D&C Red No. 18, D&C Red No. 19, D&C Red No. 117, D&C Red No. 119, D&C Red No. 121, D&C Red No. 122, D&C Red No. 127, D&C Red No. 128, D&C Red No. 130, D&C Red No. 131, D&C Red No. 134, D&C Red No. 139, FD&C Red No. 140, D&C Violet No. 12, D&C Yellow No. 17, Ext. D&C Yellow No. 17, D&C Yellow No. 18, D&C Yellow No. 111, D&C Brown No. 11, Ext. D&C Violet No. 12, D&C Blue No. 16 and D&C Yellow No. 110.

The above dyes are well known, commercially available materials, with their chemical structure being described, e.g., in 21 C. F. R. Part 74 (as revised Apr. 1, 1988) and in the CTFA Cosmetic Ingredient Handbook, (1988), published by the Cosmetics, Toiletry and Fragrances Association, Inc.

The certified dyes can be water-soluble or, preferably, lakes thereof. Lakes are organic pigments prepared by precipitating a soluble dye on a reactive or absorbent stratum, which is an essential part of the pigment's composition. Most lakes are aluminum, barium or calcium derived. These insoluble pigments are used mostly in makeup products, either powders or liquids, when a temporary color is desired that won't stain the skin (as oil-soluble dyes tend to do). The lakes are used in these products along with inorganic colors such as iron oxide, zinc oxide and titanium dioxide (the whitest white pigment).

The following tables list currently available dyes and colorants approved for use in food, drugs and/or cosmetics. The selected colorant for use herein is preferably selected from the following exemplary lists.

TABLE I
Dyes certified for use in foods, drugs, cosmetics (FDC colors)

| | | |
|---|---|---|
| FD&C Blue No. 1 | FD&C Green No. 3 | FD&C Red No. 4 |
| FD&C Red No. 40 | FD&C Yellow No. 5 | FD&C Yellow No. 6 |

TABLE 2
Dyes certified for topically applied drugs and cosmetics

| | | |
|---|---|---|
| Ext. DC Violet #2 | Ext. D&C Yellow No. 7 | Ext. D&C |
| | | Violet No. 2 |
| D&C Brown No. 1 | FD&C Red No. 4 | D&C Red No. 17 |
| D&C Red No. 31 | D&C Red No. 34 | D&C Red No. 39 |
| D&C Violet No. 2 | D&C Blue No. 4 | D&C Green No. 6 |
| D&C Green No. 8 | D&C Yellow No. 7 | D&C Yellow No. 8 |
| D&C Yellow No. 11 | D&C Orange No. 4 | D&C Orange No. 10 |
| D&C Orange No. 11 | | |

TABLE 3
Dyes certified for drugs and foods only

| | | |
|---|---|---|
| D&C Blue No. 4 | D&C Brown No. 1 | D&C Green No. 5 |
| D&C Green No. 6 | D&C Green No. 8 | D&C Orange No. 4 |
| D&C Orange No. 5 | D&C Orange No. 10 | D&C Orange No. 11 |
| D&C Red No. 6 | D&C Red No. 7 | D&C Red No. 17 |
| D&C Red No. 21 | D&C Red No. 22 | D&C Red No. 27 |
| D&C Red No. 28 | D&C Red No. 30 | D&C Red No. 31 |
| D&C Red No. 33 | D&C Red No. 34 | D&C Red No. 36 |
| D&C Violet No. 2 | D&C Yellow No. 7 | D&C Yellow No. 8 |
| D&C Yellow No. 10 | D&C Yellow No. 11 | |

Some color additives are exempt from certification and permanently listed for cosmetic use, including aluminum powder, annatto, bismuth oxychloride, bronze powder, caramel, carmine, beta-carotene, chromium hydroxide green, chromium oxide green copper (metallic powder), dihydroxyacetone, disodium EDTA-copper, ferric ammonium ferrocyanide, ferric ferrocyanide, guanine (pearl essence), guaiazulene (azulene), iron oxides, luminescent zinc sulfide, manganese violet, mica, pyrophyllite, silver (for coloring fingernail polish), titanium dioxide, ultramarines (blue, green, pink, red & violet), and zinc oxide.

The process to make the colored particles of the present invention involves dispersing the aqueous solution of matrix polymer containing a colorant into a water-immiscible liquid. Typically the water-immiscible liquid is an organic liquid or blend of organic liquids. The preferred organic liquid is a volatile paraffin oil but mixtures of a volatile and non-volatile paraffin oil may also be used. Mixtures of a volatile and non-volatile paraffin oil may be used in about equal proportions by weight, but generally it is preferred to use the non-volatile oil in excess, for instance greater than 50 to 75 parts by weight of the non-volatile oil to 25 to less than 50 parts by weight of the volatile oil.

In the process it is desirable to include a polymeric amphipathic stabilizer in the water-immiscible liquid. The amphipathic stabilizer may be any suitable commercially available amphipathic stabilizer, for instance HYPERMER® (available from ICI). Suitable stabilizers also include the stabilizers described in WO-A-97/24179.

Although it is possible to include other stabilizing materials in addition to the amphipathic stabilizer, such as surfactants, it is generally preferred that the sole stabilizing material is the amphipathic stabilizer.

In the process the dehydration step can be achieved by any convenient means. Desirably subjecting the water-in-oil dispersion to vacuum distillation can effect dehydration. Generally this will require elevated temperatures, for instance temperatures of 25° C. or higher. Although it may be possible to use much higher temperatures e.g. 80 to 90° C., it is generally preferred to use temperatures of below 70° C., for example 30 to 60° C.

Instead of vacuum distillation it may be desirable to effect dehydration by spray drying. Suitably this can be achieved by the spray drying process described in WO-A-97/34945.

The dehydration step removes water from the aqueous solution in the matrix polymer and also the volatile counterion component, resulting in a dry polymer matrix, which is insoluble and non-swellable in water, containing therein the colorant, which is distributed throughout the polymeric matrix.

Encapsulated colorant microparticles having average diameters of 0.1 to 60 microns are preferred for skin care applications, for example 1 to 40 and especially 1 to 30 microns. The encapsulated colorant microparticles may comprise 1 to 60% by weight of at least one colorant, for example 5-40% and especially 7 to 25% by weight.

Depending on the intended use, the preferred average diameters will vary. For example one embodiment of this invention may be a liquid facial skin care formulation comprising at least 2 encapsulated colorants and having a preferred range of particle sizes of between 10 and 30 microns. Another embodiment may be a lipstick formulation comprising at least 2 encapsulated colorants having preferred particle sizes of between 1 and 10 microns.

In the present invention, a skin care composition and/or application and/or preparation and/or formulation is defined as compositions useful on the face, lips, eyelashes, hand, and body. It has been found that applying a skin care formulation composition comprising microparticles having at least one encapsulated colorant incorporated therein produces desirable effects upon application. Notably, the compositions containing a blend of at least 2 microencapsulated colorants having unique and distinct colors, particularly a blend of more than one primary color, are effective means for producing natural, textured skin tone effects. Additionally, the microencapsulated colorants may provide a more vibrant color to products used around the eye area, including eyelashes. The primary colors are understood to mean red, yellow and blue. An additional feature of the inventive microparticles is the elimination of milling or grinding often encountered with non-encapsulated colorants. Said colorants are preferably organic. For other skin care applications, for example a rouge or blush, the formulation may contain only one microencapsulated colorant.

In one embodiment the skin care composition comprises a blend of microencapsulated colorants that are individually provided in at least 2 separate matrix polymer materials. In another embodiment at least 2 microencapsulated colorants are present within a single polymeric matrix material.

The skin care composition according to the invention comprises from about 0.1 to about 70% by weight, for example from about 0.5 to about 50% by weight, and especially from about 0.5 to about 35% by weight based on the total weight of the composition, of at least one encapsulated colorant as well as a cosmetically tolerable carrier or adjuvant. While water is cosmetically tolerable, and in most instances will also be present, the phrase “a cosmetically tolerable carrier or adjuvant” is intended to also include substances other than water that are customarily employed in skin care compositions.

The skin care preparation according to the invention may be formulated as a water-in-oil, oil-in-water, water-in-silicone, or silicone-in-water emulsion, an alcoholic or alcohol-containing formula, a vesicular dispersion of an ionic or non-ionic amphiphilic lipid, an anhydrous liquid or solid, an aqueous liquid or solid, a gel, or a solid stick or powder. Preferably the skin care preparation is in the form of a liquid, solid, or powder.

As a water-in-oil or oil-in-water emulsion, the skin care preparation preferably contains from about 5 to about 50% of an oily phase, from about 0.5 to about 20% of an emulsifier and from about 10 to about 90% water. The oily phase may contain any oil suitable for skin care formulations, e.g. one or more hydrocarbon oils, a wax, natural oil, silicone oil, a fatty acid ester or a fatty alcohol.

Skin care liquids may include minor amounts, for example up to about 10 weight percent of mono- or polyols such as ethanol, isopropanol, propylene glycol, hexylene glycol, glycerol or sorbitol.

Skin care formulations according to the invention may be contained in a wide variety of preparations. Especially the following preparations, for example, come into consideration: skin emulsions, multi-emulsions, skin oils, body powders; facial make-up in the form of lipsticks, lip gloss, eye shadow, mascara, eyeliner, liquid make-up, pressed powder cosmetics, day creams or powders, facial lotions, creams and powders (loose or pressed); and light-protective preparations, such as sun tan lotions, creams and oils, sun blocks and pretanning preparations.

Depending upon the form of the skin care preparation, it will comprise, in addition to the microparticulate colorants, further constituents, for example sequestering agents, additional colorants and effect pigments such as pearlescents, perfumes, thickening or solidifying (consistency regulating) agents, film-forming agents, absorbents, anti-acne actives, antiperspirant actives, anti-wrinkle and anti-skin atrophy actives, astringents, hydrophilic conditioning agents, hydrophobic conditioning agents, light diffusers, oil-soluble polymeric gelling agents, hydrophilic gelling agents, crosslinked silicone polymers, desquamating agents, vitamin compounds and precursors, chelators, enzymes, flavinoids, sterol compounds, emollients, UV absorbers and sunscreen actives, skin-protective and/or skin-soothing and/or skin healing agents, antioxidants, preservatives, skin-whitening and/or skin-lightening agents and/or self-tanning agents.

Compositions according to the invention may be prepared by physically blending suitable microparticulate colorants into skin care formulations by methods that are well known in the art. The examples herein describe several such methods. The present invention also provides a method of coloring the skin/body/eyelashes that comprises application of a liquid or solid skin care formulation having an effective coloring amount of a blend of at least one encapsulated colorant as described above to at least a part of said skin/body/eyelashes.

### EXAMPLE 1

### Microparticulate Colorant Prepared Using Silicone Surface Modifier and Polymer Blend

An aqueous colorant phase was prepared by adding 7.5 g Yellow #5 Aluminum dye lake (FD&C Yellow 5 Al Lake (SunCROMA® ex Sun Chemical as supplied) to 30 g of an approximately 30% aqueous solution of a methacrylate copolymer (polymer A—methyl methacrylate-ethyl acrylate-methyl acrylate-acrylic acid 35/27/27/11 weight % monomer ratio, having a molecular weight of about 10,000) and this mixture was stirred under high shear until the colorant was well dispersed.

The colorant phase was subsequently added to a second aqueous solution comprised of 100 g of an approx 46% by weight methacrylate microemulsion polymer (polymer B—a microemulsion containing 32% by weight of a styrene-methyl methacrylate copolymer (70/30 weight % monomer ratio, having a molecular weight of about 200,000 and a 14 weight % of a styrene-acrylic acid copolymer (65/35 weight % monomer ratio, having a molecular weight of about 6,000) and 40 g of water. After an initial mix, 16 g of zinc oxide was added and the aqueous phase was mixed under high shear until all the components were well dispersed.

An oil phase was prepared by mixing 400 g of a hydrocarbon solvent (Isopar G, ex Multisol, Chester UK), 40 g of a 25% by weight hydrocarbon solution of an amphipathic polymeric stabilizer (polymer D—copolymer of stearyl methacrylate/butyl acrylate/acrylic acid 60/21/19 weight % monomer ratio, having a molecular weight of about 10,000) and 4 g Ciba TINOCARE® SiAl, an amino-functional silicone from Ciba Specialty Chemicals Corporation.

The aqueous phase was added to the oil phase while mixing with a Silverson L4R high shear laboratory mixer. The emulsion was homogenized for 20 minutes while maintaining the temperature below 30° C.

The water-in-oil emulsion was transferred to a 2000 ml reaction flask and subjected to vacuum distillation to remove the water from the microcapsules. After distillation, the microcapsule slurry in hydrocarbon solvent was filtered to remove the solvent. The filter cake was washed with water and oven dried at 90° C. to obtain a free flowing powder.

The resultant microcapsules had a dye lake loading of ˜8% by weight with an average particle size of 25 μm (measured using a Sympatec particle size analyzer).

### EXAMPLE 2

### Microparticulate Colorant Prepared Using Alternative Surface Modifier and Polymer Blend

An aqueous colorant phase was prepared by adding 7.5 g Yellow #5 lake (SunCROMA® ex Sun Chemical as supplied) to 30 g of an approximately 30% aqueous solution of a methacrylate copolymer (polymer A—as per Example 1) and this mixture was stirred under high shear until the colorant was well dispersed. The colorant phase was subsequently added to a second aqueous solution comprised of 100 g of an approximately 46% methacrylate emulsion polymer (Polymer B as per Example 1) and 40 g of water. After an initial mix, 16 g of zinc oxide was added and the aqueous phase was mixed under high shear until all of the components were well dispersed.

An oil phase was prepared by mixing 400 g of a hydrocarbon solvent (Isopar G, ex Multisol, Chester UK), 40 g of a 25% by weight hydrocarbon solution of an amphipathic polymeric stabilizer (Polymer D as per Example 1) and 8 g of octadecanol.

The aqueous phase was added to the oil phase while mixing with a Silverson L4R high shear laboratory mixer. The emulsion was homogenized for 20 minutes while maintaining the temperature below 30° C.

The resulting water-in-oil emulsion was transferred to a 700 ml reaction flask and subjected to vacuum distillation to remove the water from the microcapsules. After distillation, the microcapsule slurry in hydrocarbon solvent was filtered to remove the solvent. The filter cake was washed with water and oven dried at 90° C. to obtain a free flowing powder.

The resultant microcapsules had a dye lake loading of 8% by weight with an average particle size of 25 μm (measured using a Sympatec particle size analyzer).

### COMPARATIVE EXAMPLE 1a

### Microparticulate Colorant Prepared Using a Single Polymer

An aqueous colorant phase was prepared by adding 7.5 g Yellow #5 lake (SunCROMA® ex Sun Chemical as supplied) to 180 g of an approximately 30% by weight solution of a methacrylate polymer (Polymer A as per Example 1) and 50 g of water. After an initial mix, 16 g of zinc oxide was added and the aqueous phase was mixed under high shear until all of the components were well dispersed.

An oil phase was prepared by mixing 400 g of hydrocarbon solvent (Isopar G, ex Multisol, Chester UK), 40 g of a 25% by weight hydrocarbon solution of amphipathic polymeric stabilizer (Polymer D as per Example 1) and 4 g Ciba TINOCARE® SiAl.

The aqueous phase was added to the oil phase while mixing with a Silverson L4R high shear laboratory mixer. The emulsion was homogenized for 20 minutes while maintaining the temperature below 30° C.

The resulting water-in-oil emulsion was transferred to a 700 ml reaction flask and subjected to vacuum distillation to remove the water from the microcapsules. After distillation, the microcapsule slurry in hydrocarbon solvent was filtered to remove the solvent. The filter cake was washed with water and oven dried at 90° C. to obtain a free flowing powder.

### COMPARATIVE EXAMPLE 1b

### Microparticulate Colorant Prepared Using a Single Polymer

An aqueous colorant phase was prepared by adding 7.5 g Yellow #5 lake (SunCROMA® ex Sun Chemical as supplied) to 120 g of an approximately 45% by weight solution of a methacrylate emulsion polymer (Polymer B as per Example 1) and 50 g of water. After an initial mix, 16 g of zinc oxide was added and the aqueous phase was mixed under high shear until all of the components were well dispersed.

An oil phase was prepared by mixing 400 g of hydrocarbon solvent (Isopar G, ex Multisol, Chester UK), 40 g of a 25% by weight hydrocarbon solution of amphipathic polymeric stabilizer (Polymer D as per Example 1) and 4 g Ciba TINOCARE® SiAl.

The aqueous phase was added to the oil phase while mixing with a Silverson L4R high shear laboratory mixer. The emulsion was homogenized for 20 minutes while maintaining the temperature below 30° C.

The resulting water-in-oil emulsion was transferred to a 700 ml reaction flask and subjected to vacuum distillation to remove the water from the microcapsules. After distillation, the microcapsule slurry in hydrocarbon solvent was filtered to remove the solvent. The filter cake was washed with water and oven dried at 90° C. to obtain a free flowing powder.

### COMPARATIVE EXAMPLE 1c

### Microparticulate Colorant Prepared Using a Single Polymer

An aqueous colorant phase was prepared by adding 7.5 g Yellow #5 lake (SunCROMA® ex Sun Chemical as supplied) to 120 g of an approximately 45% by weight solution of a methacrylate emulsion polymer (Polymer B as per Example 1) and 50 g of water. After an initial mix, 16 g of zinc oxide was added and the aqueous phase was mixed under high shear until all of the components were well dispersed.

An oil phase was prepared by mixing 400 g of hydrocarbon solvent (Isopar G, ex Multisol, Chester UK), 40 g of a 25% by weight hydrocarbon solution of amphipathic polymeric stabilizer (Polymer D as per Example 1).

The aqueous phase was added to the oil phase while mixing with a Silverson L4R high shear laboratory mixer. The emulsion was homogenized for 20 minutes while maintaining the temperature below 30° C.

The resulting water-in-oil emulsion was transferred to a 700 ml reaction flask and subjected to vacuum distillation to remove the water from the microcapsules. After distillation, the microcapsule slurry in hydrocarbon solvent was filtered to remove the solvent. The filter cake was washed with water and oven dried at 90° C. to obtain a free flowing powder.

### COMPARATIVE EXAMPLE 2

### Microparticulate Colorant Prepared Using a Polymer Blend and No Modifier

The method of example 1 was followed except that the TINOCARE SiAl was omitted from the oil phase preparation.

### COMPARATIVE EXAMPLE 3

### Microparticulate Colorant Prepared Using a Non-Optimal Polymer Blend and Silicone Surface Modifier

The method of example 1 was followed except that the aqueous phase was prepared as follows:

An aqueous colorant phase was prepared by adding 7.5 g Yellow #5 Al lake (SunCROMA® ex Sun Chemical as supplied) to 90 g of an approximately 30% aqueous solution of a methacrylate copolymer (polymer A as per Example 1) and this mixture was stirred under high shear until the colorant was well dispersed. The colorant phase was subsequently added to a second aqueous solution comprised of 60 g of an approximately 46% by weight methacrylate emulsion polymer (polymer B as per Example 1) and 20 g water. After an initial mix, 16 g of zinc oxide was added and the aqueous phase was mixed under high shear until all components were well dispersed.

### COMPARATIVE EXAMPLE 4

### Microparticulate Colorant Prepared Using a Polymer Blend and a Silicone Surface Modifier Added Post Distillation

An aqueous colorant phase was prepared by adding 7.5 g Yellow #5 Al lake (SunCROMA® ex Sun Chemical as supplied) to 30 g of an approximately 30% aqueous solution of a methacrylate copolymer (Polymer A as per Example 1) and this mixture was stirred under high shear until the colorant was well dispersed. The colorant phase was subsequently added to a second aqueous solution comprised of 100 g of an approximately 46% by weight methacrylate emulsion polymer (Polymer B as per example 1) and 40 g water. After an initial mix, 16 g of zinc oxide was added and the aqueous phase was mixed under high shear until all the components were well dispersed.

An oil phase was prepared by mixing 400 g of hydrocarbon solvent (Isopar G, ex Multisol, Chester UK), 40 g of 25% by weight hydrocarbon solution of amphipathic polymeric stabilizer (Polymer D as per Example 1).

The aqueous phase was added to the oil phase while mixing with a Silverson L4R high shear laboratory mixer. The emulsion was homogenized for 20 minutes while maintaining the temperature below 30° C.

The water-in-oil emulsion was transferred to a 700 ml reaction flask and subjected to vacuum distillation to remove the water from the microcapsules. After distillation 4 g of Ciba TINOCARE® SiAl was added and the mixture held at 90° C. for an hour.

Once cooled, the microcapsule slurry in hydrocarbon solvent was filtered to remove the solvent. The filter cake was washed with water and oven dried at 90° C. to obtain a free flowing powder.

### Bleed Test Methodology.

Bleed test solutions were prepared: 1) pH 4 citrate buffer (FIXANAL), 2) pH 7 phosphate buffer (FIXANAL), 3) aqueous solution of 5% by weight propylene glycol (pg), and 4) aqueous solution of 5% by weight of Tween 80. The test was performed by adding 1 g of microparticulate colorant to 99 g of each testing medium. This test solution was shaken and placed in an oven at 40° C. for 24 hours. Once cooled, the aqueous liquor was passed through a sub-micron Millipore filter to remove any capsule debris prior to being visually assessed against colorant standards previously prepared by progressive dilution of a solution of the same dye of known concentration in parts per million (ppm). Alternatively, dye concentrations in aqueous liquor may be analyzed by absorption spectroscopy at the corresponding wavelength of interest and determined accordingly using a Beer's Law calibration, a procedure familiar to those skilled in the art. All products in the table below used FD&C Yellow #5 as the colorant.

| | | | | | | Bleed results (ppm) | | | |
|---|---|---|---|---|---|---|---|---|---|
| Example | Polymer | Polymer | Modifier | | | | | 5% | 5% Tween |
| Number | A, wt % | B, wt % | Type³ | Wt %¹ | Stage² | pH 4 | pH 7 | pg | 80 |
| CEX 1c | 0 | 100 | None | — | — | 25 | 25 | 25 | 25 |
| CEX 1a | 100 | 0 | SiA1 | 1 | Pre | 25 | 10 | 10 | 25 |
| | | | | | dehyd. | | | | |
| CEX 1b | 0 | 100 | SiA1 | 1 | Pre | 25 | >100 | 50 | >100 |
| | | | | | dehyd. | | | | |
| CEX 2 | 15 | 85 | None | — | — | 25 | 25 | 10 | 5 |
| EX 1 | 15 | 85 | SiA1 | 2 | Pre | <1 | 0 | <1 | <1 |
| | | | | | dehyd. | | | | |
| CEX 3 | 50 | 50 | SiA1 | 2 | Pre | 50 | >100 | 50 | 50 |
| | | | | | dehyd. | | | | |
| EX 2 | 15 | 85 | StOH | 2 | Pre | 0 | 5 | 0 | 0 |
| | | | | | dehyd. | | | | |
| CEX 4 | 15 | 85 | SiA1 | 1 | Post | 25 | 10 | 10 | 10 |
| | | | | | rxn. | | | | |

¹wt % is calculated on the basis of the weight of the original hydrocarbon oil.
²Stage (of introduction) indicates at which point the modifier was introduced.
³SiA1 = Ciba TINOCARE SiA1; StOH = stearyl alcohol.

The results in the table reveal the benefits of the present invention.
The moderate effect of polymer blend introduction alone can be seen by comparison of the bleed results of example CEX 1c and example CEX 2.
The limited effect of the additive alone can be seen by comparison of the bleed results for examples CEX 1c and CEX 1b.
The effect of the introduction of both the polymer blend and the additive within the reaction scheme (i.e. the embodiment according to this invention) can be seen by the comparison of the bleed results of examples EX 1 and EX 2 versus example CEX 2.
The effect of the additive type can be seen by comparison of the bleed results of examples EX 1 and EX 2. These examples show that surface modifier choice gives a small difference; both are a significant improvement over the polymer blend alone.
The effect of the introduction of the additive during the processing scheme compared to post treatment can be seen by the comparison of the bleed results of examples EX 1 and CEX 4. Post treatment of the powder with the silicone surface modifier is substantially less effective.

### SKIN CARE COMPOSITION EXAMPLES

### Example 1

Oil-in-Water Mascara Composition

| Phase | Material | Weight % |
|---|---|---|
| A | Glyceryl Monostearate | 5.250 |
| A | Black Iron Oxide | 7.250 |
| A | Disteardimonium Hectorite | 2.250 |
| A | Stearic Acid | 2.750 |
| A | Carnauba Wax | 2.000 |
| A | Triethanolamine | 1.750 |
| A | Synthetic Wax | 1.500 |
| A | Polyvinyl Alcohol | 1.500 |
| A | Propylene Carbonate | 0.750 |
| A | Lecithin | 1.250 |
| A | Oleic Acid 80% | 1.000 |
| B | Acrylates Copolymer | 5.170 |
| B | Deionized Water | 40.18 |
| B | Simethicone Emulsion 30% | 0.200 |
| C | Xanthan Gum | 0.6 |
| C | Propylene Glycol | 3.000 |
| D | Ammonium Acrylates Copolymer | 17.79 |
| E | Ethyl Alcohol SD 40-B | 1.000 |
| E | Benzyl Alcohol | 0.650 |
| E | Panthenol | 0.280 |
| E | Phenoxyethanol | 0.280 |
| E | Methylparaben | 0.200 |
| E | Ethylparaben | 0.200 |
| E | Propylparaben | 0.100 |
| E | Trisodium EDTA | 0.100 |
| F | Encapsulated Red Pigment | — |
| F | Encapsulated Yellow Pigment | — |
| F | Encapsulated Blue Pigment | 3.00 |
| TOTAL | | 100.000 |

Phase A is heated to melt the waxes and allow the pigment to be dispersed with a Cowles Blade mixer. Phase B materials are stirred together at ambient conditions, and Phase C materials are stirred together at ambient conditions and then it is added to Phase B (to gel Phase B), and the mixture is stirred and then heated to about 85° C. The Phase A and Phases B/C are mixed together to create an oil (wax) in water emulsion. The mixture is stirred for 15 minutes and then is cooled gradually to room temperature. During the cool down, Phases D and E are added to the mixture and stirred in below 60° C. Phase F is added to and mixed with the mascara once the mascara has cooled down to about 25-50° C.

### Example 2

Oil-in-Water Mascara Composition

| Phase | Material | Weight % |
|---|---|---|
| A | Glyceryl Monostearate | 5.250 |
| A | Black Iron Oxide | 7.250 |
| A | Disteardimonium Hectorite | 2.250 |
| A | Stearic Acid | 2.750 |
| A | Carnauba Wax | 2.000 |
| A | Triethanolamine | 1.750 |
| A | Synthetic Wax | 1.500 |
| A | Polyvinyl Alcohol | 1.500 |
| A | Propylene Carbonate | 0.750 |
| A | Lecithin | 1.250 |
| A | Oleic Acid 80% | 1.000 |
| B | Acrylates Copolymer | 5.170 |
| B | Deionized Water | 40.18 |
| B | Simethicone Emulsion 30% | 0.200 |
| C | Xanthan Gum | 0.6 |
| C | Propylene Glycol | 3.000 |
| D | Ammonium Acrylates Copolymer | 17.79 |
| E | Ethyl Alcohol SD 40-B | 1.000 |
| E | Benzyl Alcohol | 0.650 |
| E | Panthenol | 0.280 |
| E | Phenoxyethanol | 0.280 |
| E | Methylparaben | 0.200 |
| E | Ethylparaben | 0.200 |
| E | Propylparaben | 0.100 |
| E | Trisodium EDTA | 0.100 |
| F | 20 μm Polyethylene Wax Particle | 1.00 |
| G | Encapsulated Red Pigment | 1.00 |
| G | Encapsulated Blue Pigment | 1.00 |
| TOTAL | | 100.000 |

Phase A is heated to melt the waxes and allow the pigment to be dispersed with a Cowles Blade mixer. Phase B materials are stirred together at ambient conditions, and Phase C materials are stirred together at ambient conditions and then it is added to Phase B (to gel Phase B), and the mixture is stirred and then heated to about 85° C. The Phase A and Phases B/C are mixed together to create an oil (wax) in water emulsion. The mixture is stirred for 15 minutes and then is cooled gradually to room temperature. During the cool down, Phases D and E are added to the mixture and stirred in below 60° C. Phase F is spherical polyethylene wax particles that are prepared separately using a typical process known in the art such as spray drying. Phases F and G are added to and mixed with the mascara once the mascara has cooled down to about 25° C.

### Example 3

Anhydrous Mascara

| Phase | Material | Weight % |
|---|---|---|
| A | Tall Oil Glycerides | 0.500 |
| A | Pentaerythrityl Hydrogenated Rosinate | 2.000 |
| A | Carnauba Wax | 5.000 |
| A | Polyethylene Wax | 8.500 |
| A | Trihydroxystearin | 2.500 |
| A | Propylparaben | 0.100 |
| A | BHA | 0.100 |
| A | Phenoxyethanol 99% | 0.800 |
| A | Petroleum Distillates | 60.50 |
| B | Disteardimonium Hectorite | 6.000 |
| C | Propylene Carbonate | 2.000 |
| D | Black Iron Oxide | 7.000 |
| E | Encapsualted Blue Pigment | 5.000 |
| TOTAL | | 100.000 |

Phase A ingredients are melted and mixed together with low shear mixing. Phase B is gradually added to the Phase A and then dispersed with high shear mixing. Phase C is then added and mixed in with high shear mixing. The Phase D is then added and dispersed with high shear mixing. The batch is cooled to ambient conditions and the Phase E is added and mixed in.

### Examples 4-5

Silicone in water facial makeup composition

| | | Example 4 | Example 5 |
|---|---|---|---|
| Phase | Material | Weight % | Weight % |
| A1 | Decamethylcyclopentasiloxane | 9.145 | 9.145 |
| A2 | Dodecamethyl cyclohexasiloxane | 2.065 | 2.065 |
| A3 | Tridecyl Neopentanoate | 8.000 | 8.000 |
| A4 | PCA Dimethicone | 2.000 | 2.000 |
| A5 | Propylparaben | 0.150 | 0.150 |
| A6 | Arachadyl Behenate | 0.300 | 0.300 |
| A7 | Stearyl Alcohol | 0.750 | 0.750 |
| B1 | Titanium Dioxide (And) Polyglyceryl-4 Isostearate (And) | 9.075 | 9.075 |
| | Cetyl Dimethicone Copolyol (And) Hexyl Laurate (And) | | |
| | Isopropyl Titanium Triisostearate (1) | | |
| B2 | Iron Oxide (CI 77492) (And) Polyglyceryl-4 Isostearate | 0.810 | 0.810 |
| | (And) Cetyl Dimethicone Copolyol (And) Hexyl Laurate | | |
| | (And) Isopropyl Titanium Triisostearate (1) | | |
| B3 | Iron Oxide (CI 77491) (And) Polyglyceryl-4 Isostearate | 0.262 | 0.262 |
| | (And) Cetyl Dimethicone Copolyol (And) Hexyl Laurate | | |
| | (And) Isopropyl Titanium Triisostearate (1) | | |
| B4 | Iron Oxide (CI 77499) (And) Polyglyceryl-4 Isostearate | 0.143 | 0.143 |
| | (And) Cetyl Dimethicone Copolyol (And) Hexyl Laurate | | |
| | (And) Isopropyl Titanium Triisostearate (1) | | |
| B5 | Silica (2) | — | 2.00 |
| C1 | Deionized Water | 54.000 | 52.000 |
| C2 | Methylparaben | 0.200 | 0.200 |
| C3 | Phenoxyethanol | 0.500 | 0.500 |
| C4 | Hydroxypropyl Starch Phosphate | 2.000 | 2.000 |
| C5 | Glycerin | 2.250 | 2.250 |
| C6 | Butylene Glycol | 2.250 | 2.250 |
| C7 | Polyvinylpyrrolidone | 1.000 | 1.000 |
| C8 | Trisodium Edetate | 0.100 | 0.100 |
| C9 | Sucrose Palmitate (and) Glyceryl Stearate (and) Glyceryl | 2.000 | 2.000 |
| | Stearate Citrate (and) Sucrose (and) Mannan (and) Xanthan | | |
| | Gum (3) | | |
| C10 | Encapsulated Red Pigment | 1.000 | 1.000 |
| C11 | Encapsulated Yellow Pigment | 1.500 | 1.500 |
| C12 | Encapsulated Blue Pigment | 0.500 | 0.500 |

(1) Kobo Products - ITT Coated Pigments
(2) Kobo Products - Silica Shells
(3) Uniquima - Arlatone V-175

Combine ingredients C1 and C9 with maximum propeller. Add Phase C ingredients 2, 3, 5, 6, 7, 8, 10, 11, and 12. Provide maximum prop mixer blending without air incorporation. Heat Phase C to 70-80° C. Once batch reaches 70-80° C. add 50% C4. Add Phase A ingredients 1-5 to separate vessel and begin homogenizing batch. Heat Phase A to 70-80° C. Add Phase B to Phase A shear on HIGH for approximately 20-30 minutes. Once Phase AB reaches 70-80° C. add Phase A ingredients 6-7. Transfer Phase AB to Phase C while prop mixing. Blend until uniform in appearance. Homogenize batch with high shear. Add remaining 50% C4. Maintain until uniformity is achieved.

### Examples 6-7

Facial makeup

| | Example 6 | Example 7 |
|---|---|---|
| Material | Weight % | Weight % |
| DC9040 cross linked elastomer gel (1) | 25.0 | |
| KSG15 cross linked elastomer gel (2) | | 25.0 |
| Dimethicone copolyol cross-polymer (KSG21) (2) | 0.5 | 0.5 |
| Decamethylcyclopentasiloxane (DC245) (1) | 11.0 | 9.0 |
| PEG/PPG18/18 Dimethicone & | 2.0 | 2.0 |
| Cyclomethicone (DC5185) (1) | | |
| Octyl Methoxy cinnamate | 2.0 | 2.0 |
| Diethylhexyl carbonate (Tegosoft DEC) (3) | 2.0 | 2.0 |
| Fibril coated sunscreen grade Titanium dioxide | 5.5 | 5.5 |
| 50% dispersion in D5 SAS/TT0-S-3/D5 (4) | | |
| Pigmentary Titanium dioxide (9729) | 9.00 | 9.00 |
| Coated with 2% methicone (5) | | |
| Black Iron Oxide coated with 2% methicone (5)* | 0.12 | 0.12 |
| Yellow Iron Oxide coated | 1.2 | 1.2 |
| with 2% methicone (5)* | | |
| Red Iron Oxide coated with 2% methicone (5)* | 0.4 | 0.4 |
| Encapsulated Red Pigment | 1.25 | 0.25 |
| Encapsulated Yellow Pigment | 1.25 | 0.25 |
| Encapsulated Blue Pigment | 0.50 | — |
| Propylparabens | 0.1 | 0.1 |
| Ethylparabens | 0.1 | 0.1 |
| Methylparabens | 0.1 | 0.1 |
| Disodium EDTA | 0.1 | 0.1 |
| Benzyl alcohol | 0.25 | 0.25 |
| Sodium chloride | 2.00 | 2.00 |
| Glycerin | 10.00 | 10.00 |
| Water | qs | qs |

(1) Available from Dow Corning
(2) Available from Shin-Etsu
(3) Available from Degussa
(4) Available from Miyoshi Kasei
(5) Available from Sensient
*these are added as slurries in cyclopentasiloxane (D5)

Examples 6-7 are prepared as follows: in a suitable vessel, water, glycerine, disodium EDTA and benzyl alcohol are added and mixed using conventional technology until a clear water phase is achieved. When the water phase is clear, the methylparabens are added and mixed again until clear. The resultant phase is mixed with a Silverson SL2T or similar equipment on high speed (8,000 rpm, standard head). In a separate vessel, the KSG21, DC245, Pigment dispersion, encapsulated red, yellow, and blue pigments, other oils, dispersant and the parabens are added and the mixture is milled using a Silverson SL2T on a high speed setting until a homogeneous mixture is created.

Following this step, the water phase and the silicone phase are combined and milled using the Silverson SL2T on a high speed setting until the water is fully incorporated and an emulsion is formed. The elastomer is then added and the mixture is mixed again using the Silverson on a high speed setting to generate the final product.

### Examples 8-9

Water in silicone facial makeup

| | | Example 8 | Example 9 |
|---|---|---|---|
| Phase | Material | Weight % | Weight % |
| A1 | Cyclopentacyloxane and dimethicone copolyol | 9.000 | 9.000 |
| A2 | Tridecyl Neopentanoate | 6.300 | 6.300 |
| A3 | Decamethylcyclopentacyloxane | 14.543 | 14.543 |
| A4 | Polyethylene Glycol (7) Lauryl Ether | 0.500 | 0.500 |
| A5 | Propylparaben | 0.150 | 0.150 |
| B1 | Titanium Dioxide (And) Polyglyceryl-4 Isostearate | 12.062 | 12.062 |
| | (And) Cetyl Dimethicone Copolyol (And) Hexyl | | |
| | Laurate (And) Isopropyl Titanium Triisostearate (1) | | |
| B2 | Iron Oxide (CI 77492) (And) Polyglyceryl-4 Isostearate | 1.382 | 1.382 |
| | (And) Cetyl Dimethicone Copolyol (And) Hexyl | | |
| | Laurate (And) Isopropyl Titanium Triisostearate (1) | | |
| B3 | Iron Oxide (CI 77491) (And) Polyglyceryl-4 Isostearate | 0.314 | 0.314 |
| | (And) Cetyl Dimethicone Copolyol (And) Hexyl | | |
| | Laurate (And) Isopropyl Titanium Triisostearate (1) | | |
| B4 | Iron Oxide (CI 77499) (And) Polyglyceryl-4 Isostearate | 0.189 | 0.189 |
| | (And) Cetyl Dimethicone Copolyol (And) Hexyl | | |
| | Laurate (And) Isopropyl Titanium Triisostearate (1) | | |
| B5 | Aluminum Starch Octenylsuccinate(4) | 5.000 | 5.000 |
| C1 | Deionized water | 17.510 | 17.510 |
| C2 | Polyvinylpyrrolidone | 1.500 | 1.500 |
| C3 | Phenoxyethanol | 0.250 | 0.250 |
| C4 | Trisodium edetate Edetate | 0.100 | 0.100 |
| C5 | Sodium Chloride | 1.000 | 1.000 |
| C6 | Sodium dehydroacetate monohydrate | 0.200 | 0.200 |
| D1 | Cyclopentasiloxane (and) C30–45 Alkyl Cetearyl | — | 12.000 |
| | Dimethicone Crosspolymer (2) | | |
| D2 | Cyclopentasiloxane (and) C30–45 Alkyl Cetearyl | 30.000 | 18.000 |
| | Dimethicone Crosspolymer (and) titanium dioxide (and) | | |
| | iron oxides (3) | | |

(1) Kobo Products - ITT Coated Pigments
(2) General Electric Silicones - Velvesil 125
(3) General Electric Silicones - 1111-21-937
(4) National Starch - Dry Flo Elite BN

Combine Phase C in plastic bucket. Provide maximum prop mixer blending without air incorporation. Add Phase A ingredients 1-6 to stainless steel jacketed vessel and begin high shear mixing. Add Phase B ingredients to Phase A and begin milling on HIGH for approximately 30 minutes. Add phase C to phase AB in vessel with homogenization. Continue homogenizing until batch uniformity is visually achieved. Add Phase D ingredients and homogenize until uniformity is achieved

### Examples 10-14

Silicone elastomer containing facial makeup

| | | Example | | | | |
|---|---|---|---|---|---|---|
| | | 10 | 11 | 12 | 13 | 14 |
| Phase | Material | Wt % | Wt % | Wt % | Wt % | Wt % |
| A | Cyclopentasiloxane | 21.64 | 33.64 | 18.14 | 12.75 | 14.30 |
| A | DC9040 Silicone Elastomer | 40.00 | 25.00 | 40.00 | 30.00 | 30.00 |
| | Gel | | | | | |
| A | KSG-21 Elastomer Gel | 5.00 | 5.00 | 5.00 | 5.00 | 5.00 |
| B | Polysilicone-7 | 0.00 | 0.00 | 0.00 | 17.39 | 17.39 |
| B | Allyl Methacrylates | 0.00 | 0.00 | 0.00 | 1.50 | 1.50 |
| | Crosspolymer | | | | | |
| | Stearyl Dimethicone | 0.00 | 3.00 | 0.00 | 0.00 | 0.00 |
| A | Titanium Dioxide & Mineral | 9.90 | 9.90 | 9.90 | 9.90 | 0.00 |
| | Oil & Methicone | | | | | |
| A | Titanium Dioxide and | 0.00 | 0.00 | 0.00 | 0.00 | 8.25 |
| | Triethylcaprylsilane | | | | | |
| B | Preservatives | 0.30 | 0.30 | 0.30 | 0.30 | 0.30 |
| A | Pigments | 3.16 | 3.16 | 3.16 | 3.16 | 3.26 |
| A | Encapsulated Red Pigment | 1.25 | 0.25 | 1.25 | 0.25 | 1.25 |
| A | Encapsulated Yellow Pigment | 1.25 | 0.15 | 1.25 | 0.25 | 1.25 |
| A | Encapsulated Blue Pigment | 0.50 | 0.10 | 0.50 | 0.00 | 0.50 |
| B | Butyl | 0.00 | 0.00 | 1.00 | 0.00 | 0.00 |
| | Methoxydibenzoylmethane | | | | | |
| B | Octyl Salicylate | 0.00 | 0.00 | 2.00 | 0.00 | 0.00 |
| B | Octocrylene | 0.00 | 0.00 | 0.50 | 0.00 | 0.00 |
| | Water | 9.55 | 9.55 | 8.33 | 16.55 | 16.55 |
| | Phenylbenzimidazole | 0.00 | 0.00 | 0.60 | 0.00 | 0.00 |
| | Sulphonic Acid | | | | | |
| | Triethanolamine | 0.00 | 0.00 | 0.62 | 0.00 | 0.00 |
| | Glycerin | 7.00 | 9.50 | 7.00 | 2.50 | 0.00 |
| | Methyl Paraben | 0.10 | 0.10 | 0.10 | 0.10 | 0.10 |
| | Disodium EDTA | 0.10 | 0.10 | 0.10 | 0.10 | 0.10 |
| | Benzyl Alcohol | 0.25 | 0.25 | 0.25 | 0.25 | 0.25 |

In a suitable stainless steel vessel, mix the phase A ingredients until homogeneous. In a separate vessel equipped with a heat source, heat the water phase materials to 50° C. and mix until homogeneous. Add the sunscreen materials, preservatives, film formers and particulates (phase B) to the batch and mix to homogeneity. If using solidifying agents, heat the cyclopentasiloxane mixture to a temperature required to melt the solidifying agents and add the solidifying agents. Cool both the water phase and silicone phase to below 30° C. and mix under high shear to form an emulsion.

### Example 15

Silicone-in-water Liquid Make-up composition

| Phase | Material | Weight % |
|---|---|---|
| A | Cyclopentasiloxane | 15.50 |
| A | Cyclohexasiloxane | 2.00 |
| A | PCA Dimethicone | 2.00 |
| A | Tridecyl Neopentanoate | 7.00 |
| A | Propylparaben | 0.20 |
| A | Methyl Methacrylate Copolymer | 2.00 |
| A | Red Iron Oxide & Isopropyl Titanuim Triisostearate | 0.27 |
| A | Yellow Iron Oxide & Isopropyl Titanium Triisosterate | 1.45 |
| A | Black Iron Oxide & Isopropyl Titanium Triisostearate | 0.21 |
| A | Anatase TiO2 & Isopropyl Titanium Triisostearate | 4.08 |
| A | Rutile TiO2 & Isopropyl Titanium Triisostearate. | 4.08 |
| A | Talc & Isopropyl Titanium Triisostearate | 0.91 |
| B | Water | 52.65 |
| B | Butylene Glycol | 0.60 |
| B | PEG-10 Dimethicone | 2.00 |
| B | Glycerin | 0.60 |
| B | Methylparaben | 0.30 |
| B | Trisodium EDTA | 0.10 |
| C | Encapsulated Yellow 5 Aluminum Lake | 0.30 |
| C | Encapsulated Red 36 | 0.20 |
| D | Phenoxyethanol | 0.80 |
| E | Hydroxethylacrylate/Sodium Acrylodimethyl | 2.75 |
| | Taurate Copolymer & Squalane & Polysorbate 60 | |
| | Total | 100.00 |

Phase A is mixed and milled together with a high shear mixer to disperse the Inorganic pigments (temperature of phase A should be kept below 50° C.). In parallel, phase B is mixed and heated to 40° C. to dissolve the Methylparaben. Once the parabens have dissolved, the encapsulated organic colorants (phase C) are added to Phase B. Phase A is then slowly added to the combined B & C phases while mixing with a high shear mixer to create a silicone-in-water emulsion. Continue emulsifying with the high shear mixer for an additional ten minutes after Phase A has been completely transferred to ensure the emulsion is homogenous (the emulsion is cooled to 25-35° C. during the emulsification phase). Phase D is then added to the emulsion and mixed for a further five minutes before phase E is added. The phase E liquid dispersion polymer is mixed into the emulsion to build viscosity and stabilize the emulsion.

### Example 16

Multi-chromatic Silicone-in-water Liquid Make-up composition

| Phase | Material | Weight % |
|---|---|---|
| A | Cyclopentasiloxane | 15.50 |
| A | Cyclohexasiloxane | 2.00 |
| A | PCA Dimethicone | 2.00 |
| A | Tridecyl Neopentanoate | 7.00 |
| A | Propylparaben | 0.20 |
| A | Methyl Methacrylate Copolymer | 2.00 |
| A | Red Iron Oxide & Isopropyl Titanium Triisostearate | 0.27 |
| A | Yellow Iron Oxide & Isopropyl Titanium | 1.45 |
| | Triisostearate | |
| A | Black Iron Oxide & Isopropyl Titanium Triisostearate | 0.21 |
| A | Anatase TiO2 & Isopropyl Titanium Triisostearate | 4.08 |
| A | Rutile TiO2 & Isopropyl Titanium Triisostearate | 4.08 |
| A | Talc & Isopropyl Titanium Triisostearate | 0.91 |
| B | Water | 49.65 |
| B | Butylene Glycol | 0.60 |
| B | PEG-10 Dimethicone | 2.00 |
| B | Glycerin | 0.60 |
| B | Methylparaben | 0.30 |
| B | Trisodium EDTA | 0.10 |
| C | Encapsulated Yellow 5 Aluminum Lake | 2.00 |
| C | Encapsulated Red 36 | 1.00 |
| C | Encapsulate Blue 1 Aluminum Lake | 0.50 |
| D | Phenoxyethanol | 0.80 |
| E | Hydroxethylacrylate/Sodium Acrylodimethyl Taurate | 2.75 |
| | Copolymer & Squalane & Polysorbate 60. | |
| | Total | 100.00 |

Phase A is mixed and milled together with a high shear mixer to disperse the Inorganic pigments (temperature of phase A should be kept below 50° C.). In parallel, phase B is mixed and heated to 40° C. to dissolve the Methylparaben. Once the parabens have dissolved, the encapsulated organic colorants (phase C) are added to Phase B. Phase A is then slowly added to the combined B & C phases while mixing with a high shear mixer to create a silicone-in-water emulsion. Continue emulsifying with the high shear mixer for an additional ten minutes after Phase A has been completely transferred to ensure the emulsion is homogenous (the emulsion is cooled to 25-35° C. during the emulsification phase). Phase D is then added to the emulsion and mixed for a further five minutes before phase E is added. The phase E liquid dispersion polymer is mixed into the emulsion to build viscosity and stabilize the emulsion.

### Example 17

Transfer Resistant Silicone-in-water Liquid Make-up composition

| Phase | Material | Weight % |
|---|---|---|
| A | Cyclopentasiloxane | 15.50 |
| A | Cyclohexasiloxane | 2.00 |
| A | PCA Dimethicone | 2.00 |
| A | Tridecyl Neopentanoate | 7.00 |
| A | Propylparaben | 0.20 |
| A | Methyl Methacrylate Copolymer | 2.00 |
| A | Red Iron Oxide & Isopropyl Titanium Triisostearate | 0.27 |
| A | Yellow Iron Oxide & Isopropyl Titanium | 1.45 |
| | Triisostearate | |
| A | Black Iron Oxide & Isopropyl Titanium Triisostearate | 0.21 |
| A | Anatase TiO2 & Isopropyl Titanium Triisostearate | 4.08 |
| A | Rutile TiO2 & Isopropyl Titanium Triisostearate | 4.08 |
| A | Talc & Isopropyl Titanium Triisostearate | 0.91 |
| B | Water | 50.65 |
| B | PEG-10 Dimethicone | 2.00 |
| B | Glycerin | 1.20 |
| B | Methylparaben | 0.30 |
| B | Trisodium EDTA | 0.10 |
| C | Encapsulated Yellow 5 Aluminum Lake | 0.30 |
| C | Encapsulated Red 36 | 0.20 |
| D | Phenoxyethanol | 0.80 |
| E | Hydroxethylacrylate/Sodium Acrylodimethyl Taurate | 2.75 |
| | Copolymer & Squalane & Polysorbate 60. | |
| F | Acrylates/C12–22 Alkylmethacrylate | 2.00 |
| | copolymer & Water & Propylene Glycol | |
| | Total | 100.00 |

Phase A is mixed and milled together with a high shear mixer to disperse the Inorganic pigments (temperature of phase A should be kept below 50° C.). In parallel, phase B is mixed and heated to 40° C. to dissolve the Methylparaben. Once the parabens have dissolved, the encapsulated organic colorants (phase C) are added to Phase B. Phase A is then slowly added to the combined B & C phases while mixing with a high shear mixer to create a silicone-in-water emulsion. Continue emulsifying with the high shear mixer for an additional ten minutes after Phase A has been completely transferred to ensure the emulsion is homogenous (the emulsion is cooled to 25-35° C. during the emulsification phase). Phase D is then added to the emulsion and mixed for a further five minutes before phase E is added. The phase E liquid dispersion polymer is mixed into the emulsion to build viscosity and stabilize the emulsion. The phase F latexed acrylate copolymer is added to the emulsion at the end of the process.

### Example 18

Lipstick

| Phase | Material | Weight % |
|---|---|---|
| A | Castor Oil | 33.25 |
| A | Triethylhexanoin | 7.50 |
| A | Triisostearyl Trilinoleate | 15.00 |
| A | Triisostearyl Citrate | 17.50 |
| A | Candelilla Wax | 7.00 |
| A | Carnauba Wax | 1.80 |
| A | Ozokerite | 1.80 |
| A | Microcrystalline Wax | 3.50 |
| A | Hydroxylated Lanolin | 1.00 |
| A | Methylparaben | 0.20 |
| A | Propylparaben | 0.10 |
| B | Encapsulated Red Pigment | 5.70 |
| B | Encapsulated Yellow Pigment | 1.10 |
| B | Encapsulated Blue Pigment | 0.20 |
| B | Mica | 4.35 |
| TOTAL | | 100.000 |

Phase A is combined, heated to between 90-105° C., and mixed until uniform. Phase B is then added with stirring until homogenous. The temperature is maintained above 70° C. as the lipstick is poured into molds.

### Example 19

Loose Powder

| Phase | Material | Weight % |
|---|---|---|
| A | Mica | 81.45 |
| A | Polymethyl Methacrylate | 5.00 |
| A | Synthetic Wax and Corn Gluten Protein | 2.00 |
| A | Titanium Dioxide | 5.00 |
| A | Methylparaben | 0.20 |
| A | Propylparaben | 0.10 |
| A | Imidazolidinyl Urea | 0.25 |
| B | Encapsulated Red Pigment | 1.00 |
| B | Encapsulated Yellow Pigment | 5.00 |
| TOTAL | | 100.000 |

Mill together A until fully dispersed. Add B to A and blend until uniform.

### Example 20

Eyeshadow

| Material | Weight % |
|---|---|
| Mica | 75.60 |
| Zinc Stearate | 5.00 |
| Encapsulated Titanium Dioxide | 6.00 |
| Encapsulated Red Pigment | 2.00 |
| Encapsulated Blue Pigment | 0.60 |
| Methylparaben | 0.20 |
| Propylparaben | 0.10 |
| Calcium Aluminum Borosilicate | 5.00 |
| PEG-4 Diheptanoate | 5.50 |
| Total | 100.000 |

Combine ingredients and mix well. Heat to 100° C. and press at 2000 psi.

### Example 21

Pressed Powder

| Phase | Material | % w/w |
|---|---|---|
| A | Soft Talc | 32.700 |
| A | Pyrenean Silk Talc | 43.195 |
| A | Titanium Dioxide | 2.000 |
| A | Silk Mica | 10.000 |
| A | Methylparaben | 0.300 |
| A | Propylparaben | 0.100 |
| A | Sodium Dehyrdroacetate Monohydrate | 0.100 |
| A | Iron Oxide (Yellow) | 0.622 |
| A | Iron Oxide (Black) | 0.182 |
| A | Iron Oxide (Red) | 0.272 |
| A | Encapsulated Red 36 | 0.200 |
| A | Encapsulated Yellow 5 Aluminum Lake | 0.300 |
| B | Octyldodecyl Stearoyl Stearate | 2.670 |
| B | Hydrogenated Coco-glycerides | 2.670 |
| B | Silicone Oil 350 centistoke | 2.670 |
| Total | | 100.000 |

Phase A ingredients are bulk mixed in a ribbon blender or double cone blender. Once the bulk Phase A ingredients are homogenous, they are passed through a hammer mill to break up powder agglomerates and extend the inorganic pigments. In parallel, the Phase B binders are heated to 60° C. On completion of milling, Phase A is returned to the ribbon blender and the hot Phase B binders are added and mixed into the bulk powder. Once the Phase A and B mixture is homogenous, the combined powder and binder ingredients are passed through a Comil. The powder is then pressed into its final form.

All documents cited in the Background, Summary of Preferred Embodiments, and Detailed Description of Illustrative and Preferred Embodiments are, in relevant part, incorporated herein by reference; the citation of any document is not to be construed as an admission that it is prior art with respect to the present invention. To the extent that any meaning or definition of a term in this document conflicts with any meaning or definition of the term in a document incorporated herein by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising at least one colorant, wherein said colorant is entrapped in at least one microparticulate matrix polymer, wherein the entrapment of said colorant in said microparticulate matrix polymer forms a microencapsulated colorant, wherein said microparticulate matrix polymer comprises:
a. at least one first polymer formed from a mixture of monomers comprising at least one first monomer that is an ethylenically unsaturated ionic monomer and at least one second monomer that is an ethylenically unsaturated hydrophobic monomer capable of forming a homopolymer with a glass transition temperature of from about −40 to about 50° C.; and
b. at least one second polymer formed from a mixture of monomers comprising at least one first monomer that is an ethylenically unsaturated ionic monomer and at least one second monomer that is an ethylenically unsaturated hydrophobic monomer capable of forming a homopolymer with a glass transition temperature greater than about 50° C.;
wherein said microparticulate matrix polymer further comprises secondary particles distributed throughout said microparticulate matrix polymer, wherein said secondary particles comprise a hydrophobic polymer formed from at least one ethylenically unsaturated hydrophobic monomer capable of forming a homopolymer with a glass transition temperature greater than about 50° C.

2. The composition of claim 1, wherein said secondary particles further comprise additional monomers, wherein said hydrophobic polymer of said secondary particles is different than said hydrophobic polymer of said microparticulate matrix polymer.

3. The composition of claim 1 further comprising a cosmetically tolerable carrier or adjuvant.

4. The composition of claim 1 comprising at least two microencapsulated colorants, wherein said colorants forming said microencapsulated colorants are distinct from one another other.

5. The composition of claim 1, wherein said colorant is selected from the group consisting of red, yellow, and blue.

6. The composition of claim 4, wherein said microencapsulated colorants are present in at least two separate microparticulate matrix polymers.

7. The composition of claim 4, wherein said microencapsulated colorants are present in a single microparticulate matrix polymer.

8. The composition of claim 1, wherein said microencapsulated colorant is present in an amount of from about 0.1 to about 70% by weight of the composition.

9. The composition of claim 1, wherein said composition is formulated as a preparation selected from the group consisting of a water-in-oil emulsion, an oil-in-water emulsion, a water-in-silicone emulsion, a silicone-in-water emulsion, a vesicular dispersion of an ionic or non-ionic amphiphilic lipid, an anhydrous liquid, an anhydrous solid, an aqueous liquid, an aqueous solid, a gel, a solid stick, and a powder.

10. The composition of claim 1, wherein said composition is in the form of a skin care application selected from the group consisting of skin emulsions, multi-emulsions, skin oils, body powders, facial make-ups, lip creams, creams, loose powders, pressed powders, light-protective preparations, lipstick, lip gloss, polymer-containing liquid lip colors, eye shadow, mascara, eyeliner, liquid make-up, powder foundations, solid emulsion make-up, day cream, day powder, facial lotion, facial cream, and facial powder.

11. The composition of claim 1, further comprising at least one additional component selected from the group consisting of sequestering agents, additional colorants, effect pigments, film-forming agents, absorbents, anti-acne actives, antiperspirant actives, anti-wrinkle actives, anti-skin atrophy actives, astringents, hydrophilic conditioning agents, hydrophobic conditioning agents, light diffusers, oil-soluble polymeric gelling agents, hydrophilic gelling agents, crosslinked silicone polymers, desquamating agents, vitamin compounds, chelators, enzymes, flavinoids, sterol compounds, emollients, UV absorbers, sunscreen actives, skin-protective agents, skin-soothing agents, skin healing agents, antioxidants, preservatives, skin-whitening agents, skin-lightening agents, and self-tanning agents.

12. A method for cosmetic treatment of skin and/or body and/or eyelashes, comprising application of a composition according to claim 1 onto at least a part of said skin and/or body and/or eyelashes.

13. The method of claim 12, wherein said composition comprises a blend of at least two microencapsulated colorants that are present in at least two separate microparticulate matrix polymers.

14. The method of claim 12, wherein said composition is formulated as a preparation selected from the group consisting of a water-in-oil emulsion, an oil-in-water emulsion, a water-in-silicone emulsion, a silicone-in-water emulsion, a vesicular dispersion of an ionic or non-ionic amphiphilic lipid, an anhydrous liquid, an anhydrous solid, an aqueous liquid, an aqueous solid, a gel, a solid stick, and a powder.
